(19) United States
(12) Patent Application Publication
(10) Pub. No.: US 2005/0096600 A1
(43) Pub. Date: May 5, 2005

(54) Safety sleeve for hiding cannula of syringes

(76) Inventor: Hui-Chiang Lin, Chunghua Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/976,755

(22) Filed: Nov. 1, 2004

(30) Foreign Application Priority Data
Nov. 4, 2003 (CN) 200320102642X

(51) Int. Cl. A61M 5/32

(52) U.S. Cl. 604/198

(57) ABSTRACT

A safety syringe includes a sleeve movably mounted to the barrel and the barrel includes a plurality of plates each of which includes a groove defined in an outer periphery thereof. The sleeve has a first flange in an inner periphery thereof and the first flange is engaged with the grooves of the plates before injection. When the injection is completed, the sleeve can be pulled till a second flange in the inner periphery thereof the sleeve is engaged with the grooves such that the cannula is enclosed and hidden by the sleeve.

### FIELD OF THE INVENTION

The present invention relates to a safety syringe having a safety sleeve which is pulled to hide the cannula therein after injection.

### BACKGROUND OF THE INVENTION

A conventional hypodermic syringe generally includes a barrel with a locking tip on a first end thereof and a needle cannula is fixed to the locking tip. A plunger is movably received in the barrel so as to push the medicine in the barrel from the needle cannula. A protection cap is used to mount to the locking tip so as to receive the needle cannula in the cap. It will be a serious concern that the needle cannula could hurt the medical personnel during removing the protection cap from the locking tip before proceeding syringing and/or re-mounting the protection cap to the locking tip after the syringing process is finished. Besides, the discarded syringe could be mis-used by the those people who utilize the syringes, no matter whatever condition the syringes is, to inject drug into their bodies.

The present invention intends to provide a safety syringe that includes a sleeve which can be pulled to hide the cannula therein after injection so as to protect the users from being hurt by the cannula.

### SUMMARY OF THE INVENTION

The present invention relates to a syringe that comprises a barrel with a connection port at a first end thereof and a plunger with a stopper is movably inserted in a passage defined in the barrel. The connection port has a tube extending from the first end of the barrel and a plurality of plates extend from the first end of the barrel and the tube is enclosed by the plates. Each plate has a groove defined in an outer periphery thereof. A locking tip has a cannula connected thereto and is mounted to the tube of the connection port. A sleeve is movably mounted to the barrel and a first flange extends from an inner periphery of a first end of the sleeve. A second flange extends from the inner periphery of a second end of the sleeve. The first flange is removably engaged with the groove of the plates before injection and the sleeve is pulled to disengage the first flange from the groove till the second flange is engaged with the groove after the injection is completed.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is an exploded view to show the syringe of the present invention;

FIG. 2 is a cross sectional view to show the syringe of the present invention before being used;

FIG. 3 shows the head of the plate of the connection port is engaged with tow ends of the stop member on the locking tip;

FIG. 4A shows that the plunger is pushed into the barrel after injection;

FIG. 4B shows the barrel is pulled in a direction away from the sleeve;

FIG. 4C shows the second flange of the sleeve is engaged with the grooves of the plates of the connection port on the barrel;

FIG. 5 is an exploded view to show another embodiment of the syringe of the present invention;

FIG. 6 is a perspective view to show the syringe in FIG. 5;

FIG. 7 shows a sleeve is mounted to the barrel of the syringe in FIG. 5;

FIG. 8 shows the head of the plate of the connection port is engaged with tow ends of the stop member on the locking tip;

FIG. 9 shows the syringe in FIG. 5 before being used;

FIG. 10A shows that the plunger is pushed into the barrel after injection, and

FIG. 10B shows the second flange of the sleeve is engaged with the grooves of the plates of the connection port on the barrel.

### DETAILED DESCRIPTION OF THE PREFERRED EMBODIMENT

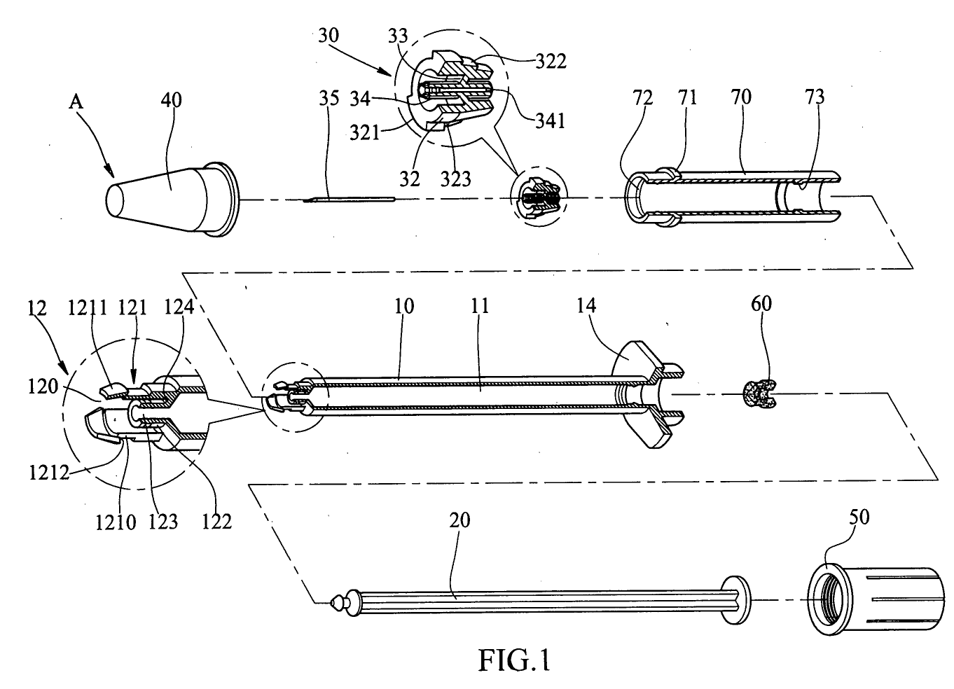

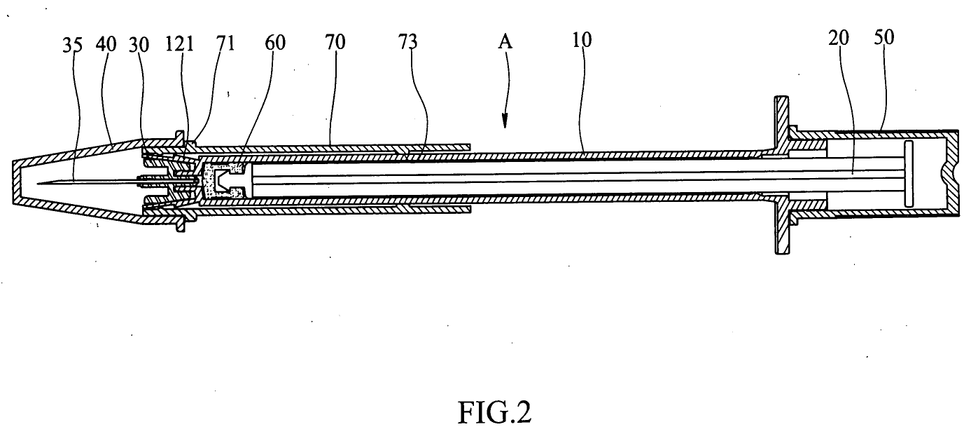

Referring to FIGS. 1 to 3, the syringe “A” of the present invention comprises a barrel 10 with a connection port 12 at a first end thereof and a finger flange 14 connected to a second end of the barrel 10. A plunger 20 has a first end connected to a stopper 60 and movably inserted in a passage 11 defined in the barrel 10 from the second end of the barrel 10. The connection port 12 has a tube 122 extending from the first end of the barrel 10 and having a through hole 123 being in communication with the passage 11 in the barrel 10. A plurality of plates 121 extend from the first end of the barrel 10 and the tube 122 is enclosed by the plates 121. Each plate 121 has a groove 1210 defined in an outer periphery thereof. Each plate 121 has a head portion 1211 which is wider than the plate 121.

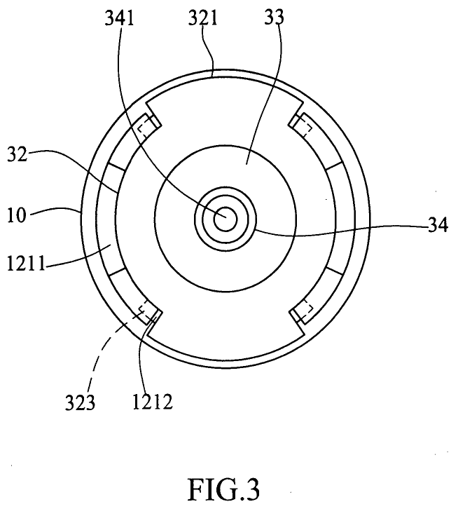

A locking tip 30 includes a tubular body and a communication tube 34 is located centrally therein and a connection board 322 is transversely connected between the communication tube 34 and an inner periphery of the tubular body. A cannula 35 is inserted in a through hole 341 defined centrally in the communication tube 34. The locking tip 30 is mounted to the tube 122 wherein the communication tube 34 is inserted in the tube 122 and a wall of the locking tip 30 is inserted in the annular groove 124 between the plates 121 and the tube 122. A plurality of blocks 321 extend outward from an outer periphery of the tubular body and are engaged with gaps 120 between the plates 121 of the connection port 12. Each block 321 is connected to a stop member 322 which is located beneath the block 321 and includes two ends 323 protruding beyond two ends of the block 321 corresponding thereto such that the two ends 1212 of the head portion 1211 are rested on the two ends of the stop member 322 as shown in FIG. 3. A protection cap 40 is mounted to the locking tip 30 so as to protect the cannula 35 from being contaminated by foreign objects and an end cap 50 is threadedly connected to the plunger 50.

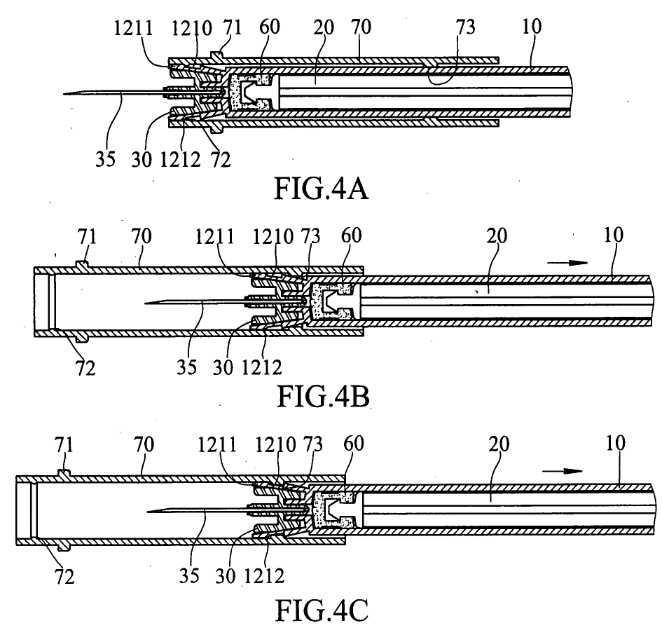

A sleeve 70 is movably mounted to the barrel 10 and a first flange 72 extends from an inner periphery of a first end of the sleeve 70. A second flange 73 extends from the inner periphery of a second end of the sleeve 70. Further referring to FIGS. 4A, 4B and 4C, the first flange 72 is removably engaged with the groove 1210 of the plates 121 before the syringe “A” is used and after the injection of the syringe “A” is completed, the user may holds the barrel 10 by one hand and a flange 71 on an outer periphery of the sleeve 70 by the other hand, and the barrel 10 is pulled in a direction away from the sleeve 70 to disengage the first flange 72 from the groove 1210 till the second flange 73 is engaged with the groove 1210. Therefore, the cannula 35 is enclosed by and hidden in the sleeve 70 and cannot hurt people.

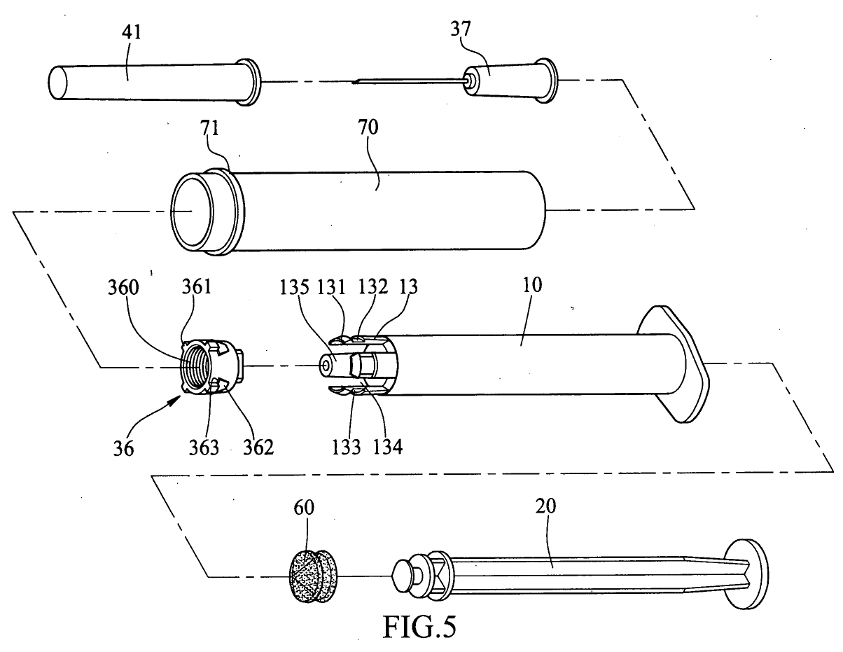

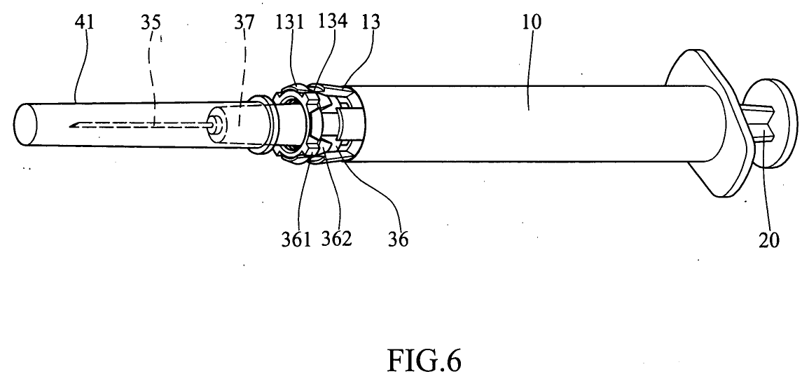

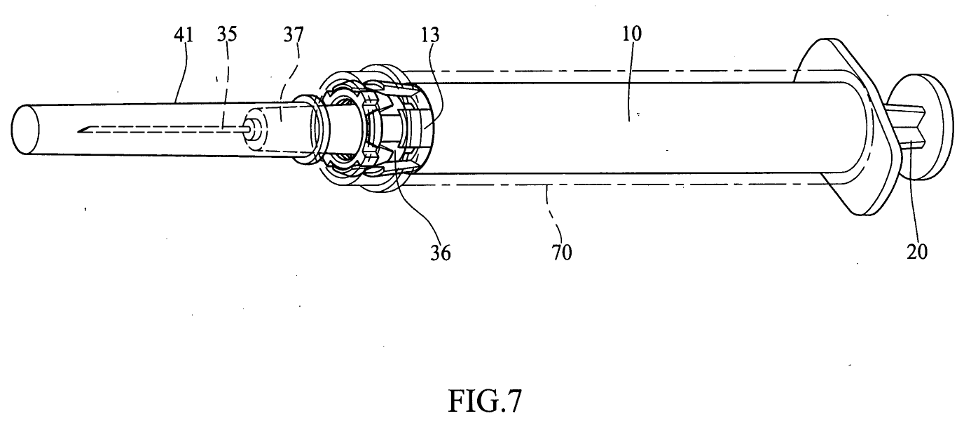

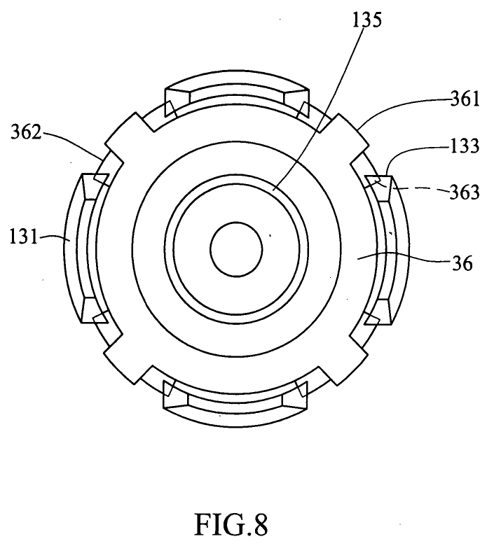

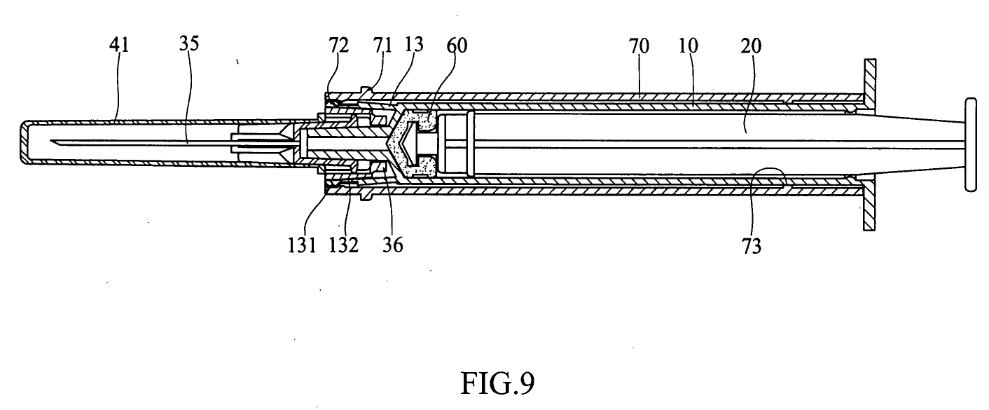

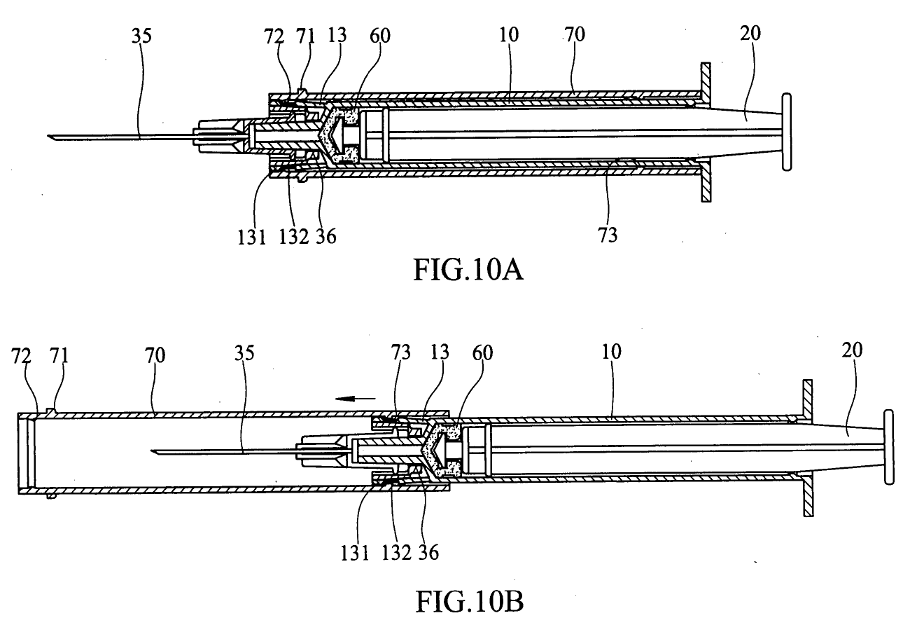

FIGS. 5 to 10A and 10B show the other embodiment of the present invention, wherein the barrel 10, the plunger 20 and the sleeve 70 are the same as the embodiment in FIG. 1, The connection port includes a plurality of plates 13 separated by gaps 134 and a tube 135 is enclosed by the plates 13. Each plate 13 includes a head portion 131 and a groove 132 is defined in an outer periphery thereof. The locking tip 36 has a threaded inner periphery 360 and the cannula 35 is connected to a base 37 which includes an annular lip which is threadedly engaged with the threaded inner periphery 360 of the locking tip 36. The locking tip 36 includes a plurality of blocks 361 and a stop member 362 is formed in an outer periphery of the locking tip 36 and below the block 361. The stop member 362 forms two ends 363 such that the two ends 133 of the head portion 131 are rested on the two ends 363 of the stop member 362. A protection cap 41 is mounted to the base 37 before the syringe is used. The first flange.

The procedure for using the sleeve 70 is the same as the embodiment in FIG. 1 and the first flange 72 of the sleeve 70 is removably engaged with the groove 132 of the plates 13 before the syringe is used and after the injection of the syringe is completed, the user may holds the barrel 10 by one hand and a flange 71 on an outer periphery of the sleeve 70 by the other hand, and the barrel 10 is pulled in a direction away from the sleeve 70 to disengage the first flange 72 from the groove 132 till the second flange 73 is engaged with the groove 132. Therefore, the cannula 35 is enclosed by and hidden in the sleeve 70 and cannot hurt people.

While we have shown and described the embodiment in accordance with the present invention, it should be clear to those skilled in the art that further embodiments may be made without departing from the scope of the present invention.

1. A syringe comprising:
a barrel with a connection port at a first end thereof and a plunger having a first end movably inserted in a passage defined in the barrel from a second end of the barrel, a stopper connected to the first end of the plunger, the connection port having a tube extending from the first end of the barrel and being in communication with the passage in the barrel, a plurality of plates extending from the first end of the barrel and the tube being enclosed by the plates, each plate having a groove defined in an outer periphery thereof;
a locking tip having a cannula connected thereto and mounted to the tube of the connection port, and
a sleeve movably mounted to the barrel and a first flange extending from an inner periphery of a first end of the sleeve, a second flange extending from the inner periphery of a second end of the sleeve, the first flange removably engaged with the groove of the plates, the sleeve being pulled to disengage the first flange from the groove till the second flange is engaged with the groove.

2. The syringe as claimed in claim 1, wherein the locking tip includes a tubular body which has a communication tube located centrally therein and a connection board is transversely connected between the communication tube and an inner periphery of the tubular body, the cannula inserted in a through hole defined centrally in the communication tube, a plurality of blocks extending outward from an outer periphery of the tubular body and engaged with gaps between the plates of the connection port.

3. The syringe as claimed in claim 2, wherein each plate has a head portion which is wider than the plate and each block is connected to a stop member which is located beneath the block and includes two ends protruding beyond two ends of the block corresponding thereto.

4. The syringe as claimed in claim 1, wherein the locking tip has a threaded inner periphery and the cannula is connected to a base which includes an annular lip which is threadedly engaged with the threaded inner periphery of the locking tip.
